(19) United States
(12) Patent Application Publication
(10) Pub. No.: US 2016/0287144 A1
(43) Pub. Date: Oct. 6, 2016

(54) IMAGE CAPTURING APPARATUS AND AUTHENTICATION APPARATUS

(71) Applicant: FUJITSU FRONTECH LIMITED, Tokyo (JP)

(72) Inventors: Isao Iwaguchi, Inagi-shi (JP); Kozo Yamazaki, Inagi-shi (JP)

(21) Appl. No.: 15/185,325

(22) Filed: Jun. 17, 2016

Related U.S. Application Data
(63) Continuation of application No. PCT/JP2014/052262, filed on Jan. 31, 2014.

(51) Int. Cl. A61B 5/1171; G06K 9/00; A61B 5/00; A61B 5/1455; A61B 5/145

(52) U.S. Cl. CPC A61B 5/1171; A61B 5/14551; A61B 5/14546; A61B 5/6826; A61B 5/6824; G06K 9/00013; A61B 5/0077; A61B 2562/16

(57) ABSTRACT

In order to capture an image of a living body with reduced noise, an image capturing apparatus includes a main body that captures a living body image, and a support attachable to and detachable from the main body. The image capturing apparatus supports the living body with a finger guide and a wrist guide of the support, and guides light emitted from the main body through the support toward its top face side from its bottom face side where the main body contacts with the support, and radiates the guided light toward the living body from a light radiating section provided at a predetermined position of an inner wall and an end portion of the top face side of the support, so that the main body captures a living body image.

### CROSS-REFERENCE TO RELATED APPLICATION

This application is a continuation application of International Application PCT/JP2014/052262 filed on Jan. 31, 2014 which designated the U.S., the entire contents of which are incorporated herein by reference.

### FIELD

The embodiments discussed herein relate to an image capturing apparatus and an authentication apparatus.

### BACKGROUND

In recent years, user authentication (biometric authentication) using biometric information for identifying each individual is becoming more prevalent. For example, biometric information utilizable in authentication is fingerprint, retina and iris of eye, vein pattern, face, blood vessel, DNA (Deoxyribo Nucleic Acid). Biometric authentication is performed by enrolling biometric information in advance and verifying biometric information acquired for verification against the biometric information enrolled in advance.

For example, biometric authentication using vein pattern is performed in financial institutions and the like. An image capturing apparatus acquires a vein pattern by radiating light to a living body and capturing an image by collecting, by means of a lens, the light that is reflected at an inner portion of the living body and exits from the living body. At this time, the light radiated to the living body is reflected on the surface of the living body and collected by the lens, depending on the incident angle of the light relative to the living body. This reflected light can be a noise that prevents capturing of an image of distinct biometric information (vein pattern) in some cases.

For example, Patent Literature 1 discloses a system that acquires biometric information by emitting light from a light source and radiating the light to a specific area of a finger. The disclosed system captures an image of a living body by radiating light to a specific area of a finger, for the purpose of preventing noise, such as light reflected on the finger surface, from being collected by a lens.

See, for example, Japanese Laid-open Patent Publication No. 2011-215665.

However, the disclosed system is sometimes unable to radiate light on a specific area of a living body, depending on the orientation of the living body at the time of capturing an image of biometric information. As a result, the disclosed system is sometimes unable to capture an image of the living body, due to noise, such as light that is reflected on a finger surface and collected by a lens.

### SUMMARY

According to an aspect, there is provided an image capturing apparatus including: a main body including an imaging unit that captures an image of a living body; and a support that contacts the main body at one side and supports the living body at a predetermined position relative to the imaging unit at another side, wherein the main body includes a light emitting unit that emits light for the living body, and the support includes: an entrance section where the light enters the one side, a light radiating section that radiates the light toward the living body at a position away from the entrance section toward the other side, and a light guiding section that guides the light from the entrance section to the light radiating section.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWING

FIG. 1 is a perspective view illustrating an exterior appearance of an image capturing apparatus in a first embodiment;

FIG. 2 is an exploded perspective view of the image capturing apparatus in the first embodiment;

FIG. 3 is a top perspective view of a main body in the first embodiment;

FIG. 4 is a cross-sectional view of the main body in the first embodiment;

FIG. 5 is a cross-sectional view of a rear wall in the first embodiment;

FIG. 6 is a cross-sectional view of the image capturing apparatus in the first embodiment;

FIG. 7 is a top perspective view of the image capturing apparatus in the first embodiment;

FIG. 8 is a cross-sectional view of a rear wall in a second embodiment;

FIG. 9 is a cross-sectional view of an image capturing apparatus in a third embodiment;

FIG. 10 is a top perspective view of a main body in the third embodiment;

FIG. 11 is a top perspective view of a main body in a fourth embodiment;

FIG. 12 is a top perspective view of an image capturing apparatus in the fourth embodiment;

FIG. 13 is an exterior view of an image capturing apparatus in a fifth embodiment;

FIG. 14 is a perspective view of an image capturing apparatus in a sixth embodiment;

FIG. 15 is a cross-sectional view of an image capturing apparatus in the sixth embodiment; and

FIG. 16 illustrates an exemplary application of an image capturing apparatus in a seventh embodiment.

### DESCRIPTION OF EMBODIMENTS

In the following, embodiments will be described with reference to drawings.

### First Embodiment

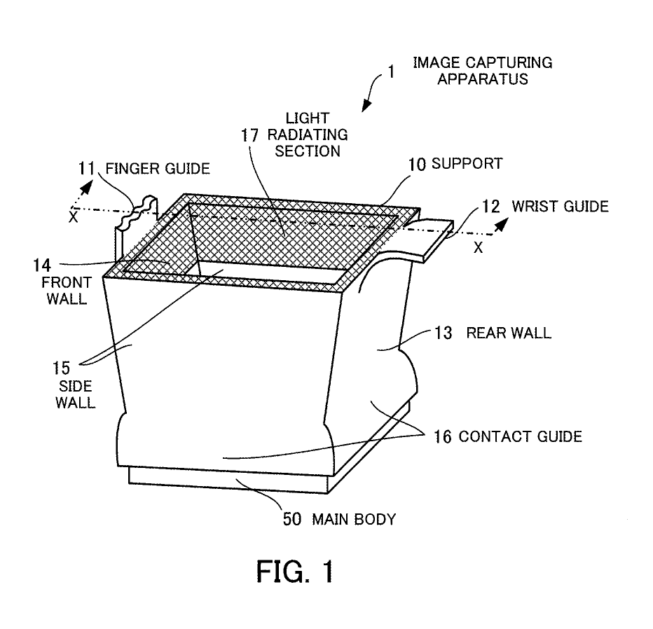

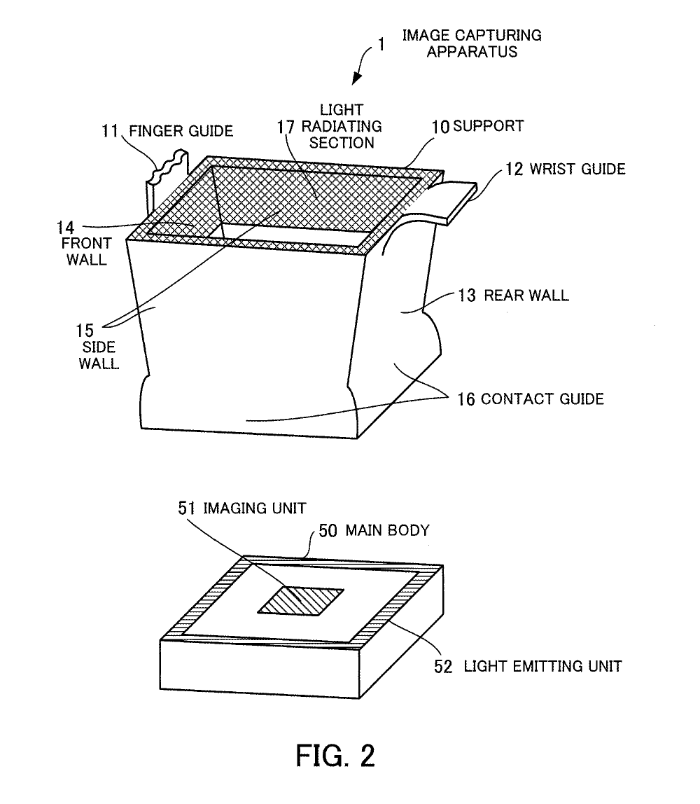

First, an image capturing apparatus of the first embodiment will be described with reference to FIGS. 1 and 2. FIG. 1 is a perspective view illustrating an exterior appearance of the image capturing apparatus in the first embodiment. FIG. 2 is an exploded perspective view of the image capturing apparatus in the first embodiment.

The image capturing apparatus 1 captures an image of a living body. The image capturing apparatus 1 captures an image of a palm (living body), particularly an image of veins in the palm. The image capturing apparatus 1 is equipped in an automatic teller machine or the like of a financial institution, for example.

The image capturing apparatus 1 captures reflection of near-infrared light from a target living body (palm). Hemoglobin in a red blood cell that flows in a vein has lost oxygen, and therefore this hemoglobin (reduced hemoglobin) has a property of absorbing near-infrared light of approximately 700 to 1000 nanometers. Hence, when near-infrared light is radiated to a palm, a vein area reflects less near-infrared light, and a vein position is recognizable from the intensity of the reflected near-infrared light. The image capturing apparatus 1 uses a specific light source to capture an achromatic image, which makes it easy to extract feature information.

The image capturing apparatus 1 includes a main body 50 that captures an image of a living body, and a support 10 that is attachable to and detachable from the main body 50. The image capturing apparatus 1 supports a living body with the support 10, and guides light emitted from the main body 50 with the support 10, and radiates the guided light from the support 10 toward the living body, and captures an image of the living body which is irradiated with the light. Note that the support 10 may be fixed to the main body 50.

The main body 50 is a cuboid housing that contains an imaging unit 51 for capturing an image of living body and a light emitting unit 52 for emitting light. The imaging unit 51 is provided at the center of the housing, facing in a direction (image capturing direction) toward a living body supported by the support 10 as an image capturing target. That is, the imaging unit 51 faces in the image capturing direction toward the housing top face side. The light emitting unit 52 surrounds the circumference of the imaging unit 51 along four sides of the housing, and emits light toward the housing top face side.

The support 10 has a shape for supporting a living body in an appropriate orientation, so that the imaging unit 51 can capture an image of the living body in a stable manner. The support 10 has a box shape of a reversed truncated pyramid or a cuboid that increases its internal hollow area toward its top from its bottom that is in contact with the main body 50. In the following, the support 10 will be described as a cuboid. The bottom face of the support 10 is open at its center and in contact with the main body 50 along the periphery of the housing top face of the main body 50 when attached to the main body 50. The support 10 is open at its top face for supporting a living body. The support 10 includes a bottom face and standing walls (a rear wall 13, a front wall 14, and two side walls 15) that stand from the bottom face.

The rear wall 13 includes a wrist guide 12 that guides a wrist at an appropriate mounting position at its top face side. The front wall 14 includes a finger guide 11 that guides fingers at an appropriate mounting position at its top face side. With these wrist guide 12 and finger guide 11, the support 10 can support a living body at an appropriate position (right position) in an appropriate orientation.

The support 10 includes a contact guide 16 at the bottom face side of the rear wall 13, the front wall 14, and the two side walls 15. When the support 10 is attached to the main body 50, the contact guide 16 guides the support 10 to a right position of the main body 50, in order to restrict the movement of the support 10 in the front, rear, left, and right directions in relation to the main body 50.

Note that, if there is an obstacle directly below the contact guide 16 when the support 10 is attached to the main body 50, an operator who attaches the support 10 to the main body 50 notices the existence of the obstacle. Note that the contact guide 16 is needless to be provided along the whole circumference of the bottom face side of the rear wall 13, the front wall 14, and the two side walls 15. For example, the contact guide 16 may be provided at a part of the whole circumference, and for example may be formed in L shapes at opposing two corners.

Also, the support 10 is formed of transparent material (for example, plastic such as acrylic, or glass), so that the rear wall 13, the front wall 14, and the two side walls 15 function as light guides.

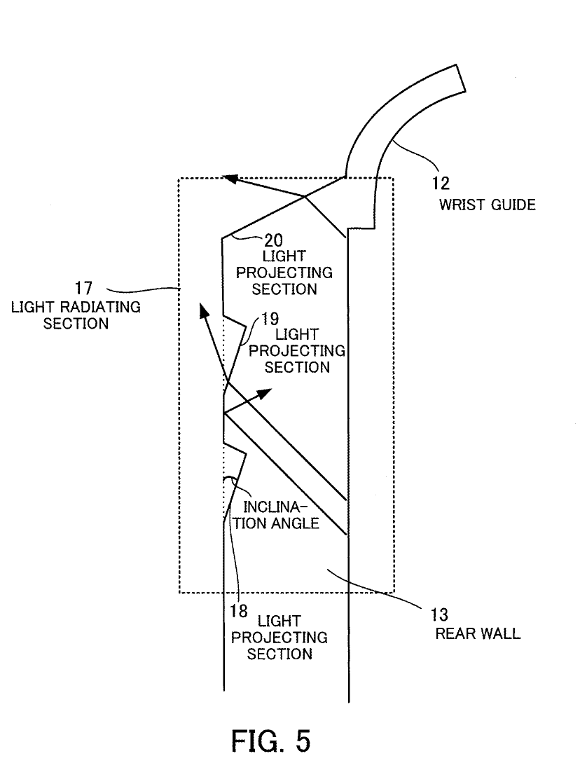

Also, the support 10 includes a light radiating section 17 that radiates light toward a living body that is supported at the top face side. The light radiating section 17 is provided at predetermined positions of the top face sides of the inner circumferential surfaces of the rear wall 13, the front wall 14, and the two side walls 15 (the surface of an inner side of a box shape), and at the top faces of the rear wall 13, the front wall 14, and the two side walls 15. The detail of the light radiating section 17 will be described later with reference to FIG. 5.

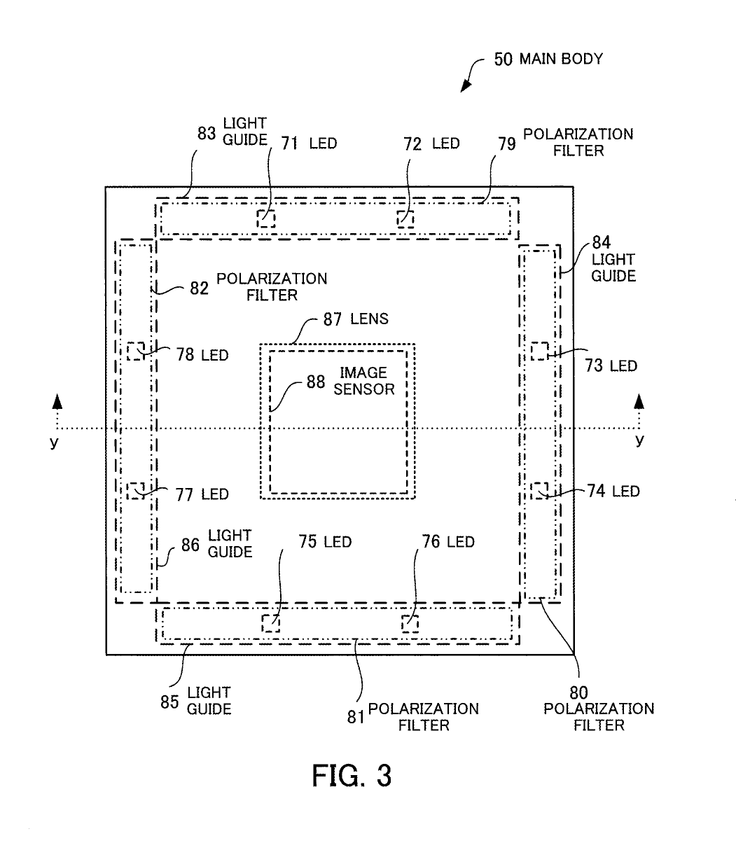

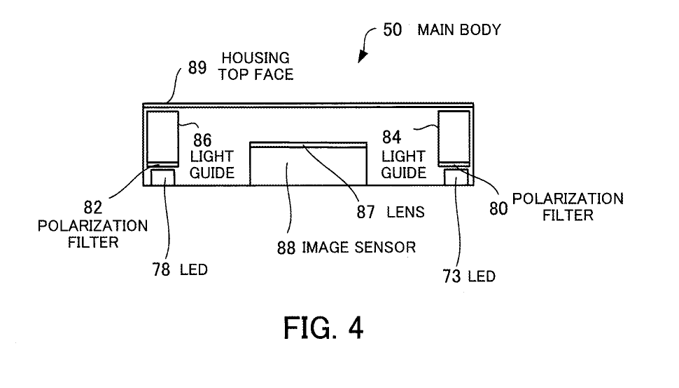

Next, the structure inside the main body 50 will be described with reference to FIGS. 3 and 4. FIG. 3 is a top perspective view of the main body in the first embodiment. FIG. 4 is a cross-sectional view of the main body in the first embodiment, in which the main body 50 of FIG. 3 is cut along the y-y line.

The main body 50 contains a lens 87, an image sensor 88, light emitting diodes (LEDs) 71 to 78, polarization filters 79 to 82, and light guides 83 to 86, in the housing. Note that the lens 87 and the image sensor 88 are components of the imaging unit 51. The image sensor 88 faces toward an image capturing target via the lens 87. The LEDs 71 to 78, the polarization filters 79 to 82, and the light guides 83 to 86 are components of the light emitting unit 52. Light emitted from the LEDs 71 to 78 exits from the housing top face and passes through the polarization filters 79 to 82 and the light guides 83 to 86.

The image sensor 88 is provided at the center of the housing. The image sensor 88 captures an image of a living body through the lens 87 provided at the housing top face side.

The LEDs 71 to 78 surround the circumference of the image sensor 88 at four sides, each of which is provided with two LEDs. The LEDs 71, 72, 75, and 76 are provided along the sides extending in the horizontal direction of the housing. The LEDs 73, 74, 77, and 78 are provided along the sides extending in the vertical direction of the housing. Note that the number of LEDs provided in the main body 50 is an example and is not limited thereto. Each of the LEDs 71 to 78 emits light to the housing top face side and ultimately to a living body.

The polarization filters 79 to 82 are provided at the housing top face side of the LEDs. The polarization filter 79 is provided at the housing top face side of the LED 71 and the LED 72. The polarization filter 80 is provided at the housing top face side of the LED 73 and the LED 74. The polarization filter 81 is provided at the housing top face side of the LED 75 and the LED 76. The polarization filter 82 is provided at the housing top face side of the LED 77 and the LED 78. Note that a polarization filter may be provided for each LED.

The polarization filters 79 to 82 receive incoming light and transmit linearly-polarized light having a vibration component in a predetermined direction. The transmission axes of the polarization filters 79 and are the vertical direction of FIG. 3. Thus, the polarization filters 79 and 81 receive incoming light and transmit linearly-polarized light having a vibration component in the horizontal direction of FIG. 3. The transmission axes of the polarization filters 80 and 82 are the horizontal direction of FIG. 3. Thus, the polarization filters 80 and 82 receive incoming light and transmit linearly-polarized light having a vibration component in the vertical direction of FIG. 3.

The light guides 83 to 86 are provided at the housing top face side of the polarization filters. The light guides 83 to 86 are provided in such a manner that their upper ends extend along the periphery of the housing top face. The light guide 83 is provided at the housing top face side of the polarization filter 79. The light guide 84 is provided at the housing top face side of the polarization filter 80. The light guide 85 is provided at the housing top face side of the polarization filter 81. The light guide 86 is provided at the housing top face side of the polarization filter 82.

The light guides 83 to 86 guide light that enters their inner portions to the housing top face side, and project the light from the end portion of the housing top face side toward the housing top face. Also, the housing top face 89 is formed of light transmissive material, such as cover glass. Thereby, the light emitting unit 52 emits the light from the periphery of the housing top face.

Note that, in the above description, the light emitting unit 52 is configured with the LEDs, the polarization filters, and the light guides, but is not limited thereto. For example, the light emitting unit 52 may be configured with LEDs only. In this case, the LEDs may be provided at the vicinity of the housing top face 89 of the main body 50. Note that the polarization filters may be provided closer to the housing top face side than the light guides. Also, the polarization filters may be provided above the lens 87.

Next, the shape of the light radiating section 17 will be described. FIG. 5 is a cross-sectional view of the rear wall of the image capturing apparatus 1 in the first embodiment, which is cut along the x-x line of FIG. 1.

The light radiating section 17 is provided at a predetermined position of the inner circumferential surface of the top face side of the rear wall 13, and at the upper end of the rear wall 13. That is, the light radiating section 17 is provided at a position away from the bottom face side of the rear wall 13 toward the top face side. The light radiating section 17 includes light projecting sections 18, 19, and 20 that project light guided from the bottom face side. The light projecting sections 18 and 19 are cutout surfaces formed on the inner circumferential surface of the rear wall 13. The light projecting section 20 is the top face of the rear wall 13. Of the light that enters the light projecting sections 18, 19, and 20, the light that does not satisfy a total reflection condition exits from the light projecting sections 18, 19, and 20.

For example, when the light projecting section is inclined at an angle of 5° from the inner circumferential surface of the rear wall 13 (when the inclination angle is 5°), the light projecting section 18 projects light that has been totally reflected and guided through the inner portion of the rear wall 13 but fails to satisfy the total reflection condition because of the inclination of 5°.

Note that the light projecting sections 18, 19, and 20 are examples and are not limited thereto. For example, the light radiating section 17 can adjust how the light is radiated to the living body, by changing the number of light projecting sections, the shapes of the light projecting sections, and the formation interval of the light projecting sections. For example, the light radiating section 17 can adjust the light amounts of light projected from the light projecting sections, by changing the lengths of the inclined portions of the light projecting sections and the formation interval of the light projecting sections. Also, the light radiating section 17 can adjust the angles of light projected from the light projecting sections and the positions at which light enters a living body, by changing the inclination angles of the light projecting sections.

Also, the light radiating section 17 can adjust how light is radiated to a living body, by changing the shapes of the light projecting sections and the formation interval of the light projecting sections, with respect to each light projecting section. Also, the light radiating section 17 may be formed on the entire area of the inner circumferential surface of the rear wall 13.

Note that the light radiating section 17 is not limited to the one including all of the light projecting sections 18, 19, and 20, but may include either the light projecting sections 18 and 19 or the light projecting section 20. Although the light radiating section 17 in the rear wall 13 has been described, the light radiating section is configured in the same way in the front wall 14 and the two side walls 15. In this case, the image capturing apparatus 1 may differentiate how light is radiated at the rear wall 13, the front wall 14, and the two side walls 15 from each other.

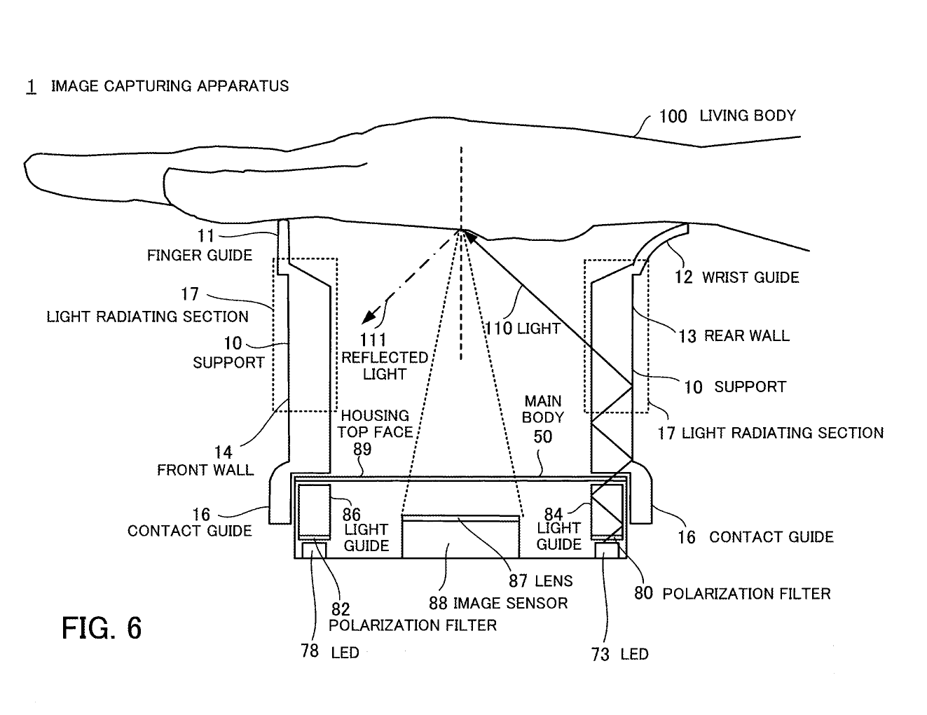

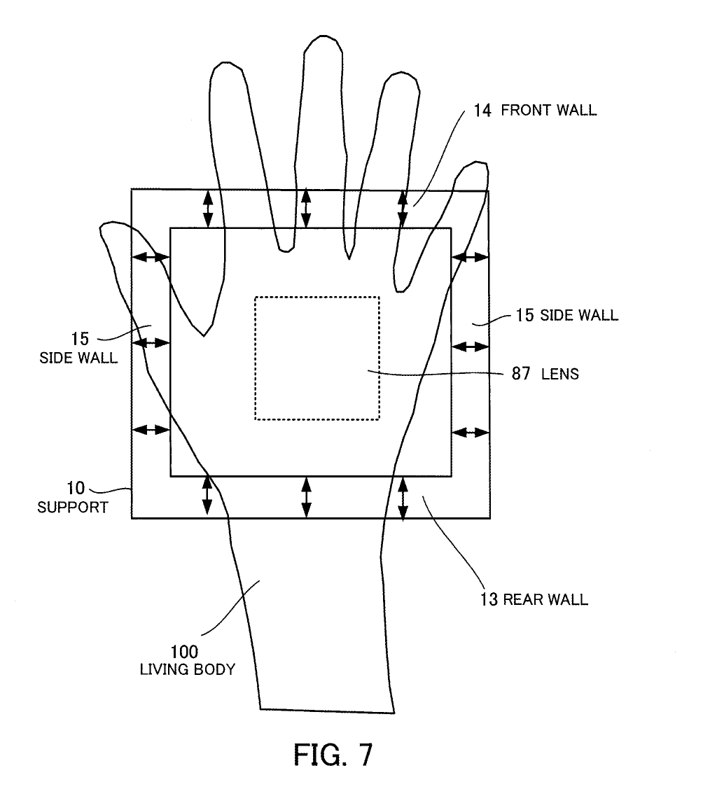

Next, guidance and radiation of light will be described with reference to FIGS. 6 and 7. FIG. 6 is a cross-sectional view of the image capturing apparatus in the first embodiment, which is cut along the x-x line of FIG. 1, with a living body mounted thereon. FIG. 7 is a top perspective view of the image capturing apparatus in the first embodiment. In the following, guidance and radiation of light will be described by taking the rear wall 13 as an example selected from among the rear wall 13, the front wall 14, and the two side walls 15.

As illustrated in FIG. 6, the LED 73 emits light 110. The light 110 emitted by the LED 73 is changed to linearly-polarized light having a horizontal vibration direction in FIG. 6 (arrow direction of FIG. 7, the thickness direction of the rear wall) to pass through the polarization filter 80. The light 110 that is changed to the linearly-polarized light enters the inner portion of the light guide 84 from the lower end portion (the end portion at the LED 73 side) of the light guide 84. After entering the inner portion of the light guide 84, the light 110 is totally reflected in the inner portion of the light guide 84 and guided to the housing top face 89 side.

After totally reflected in the inner portion of the light guide 84 and guided to the housing top face 89 side, the light 110 exits from the end portion of the housing top face 89 side of the light guide 84. After exiting from the end portion of the housing top face 89 side of the light guide 84, the light 110 passes through the housing top face 89 and exits to the outside of the main body 50. After exiting to the outside of the main body 50, the light 110 enters the inner portion of the rear wall 13 from the end portion of the bottom face side of the rear wall 13 that faces the top face of the light guide 84 with the housing top face 89 therebetween.

After entering the inner portion of the rear wall 13, the light 110 is totally reflected in the inner portion of the rear wall 13 and is guided from the bottom face side toward the top face side. After guided by the inner portion of the rear wall 13 from the bottom face side toward the top face side, the light 110 is radiated from the light radiating section 17 toward a living body 100.

A part of the light 110 radiated toward the living body 100 enters the inner portion of the living body 100. Also, a part of the light 110 radiated toward the living body 100 becomes a reflected light 111 (noise, light reflected on the surface) that hinders image capturing of the living body. The reflected light 111 reflected on a living body surface is directed to the outside of the lens 87. After entering the inner portion of the living body 100, the light 110 scatters at the inner portion of the living body 100, and exits from the living body 100, and enters the lens 87. Then, the imaging unit 51 captures an image of the living body by using the light 110 that enters the lens 87.

As described above, the image capturing apparatus 1 is configured in such a manner that light emitted from the LED 73 enters the end portion of the bottom face side of the rear wall 13 and then is guided from the bottom face side toward the top face side of the rear wall 13 and then is radiated from the light radiating section 17 toward the living body 100. Thus, the image capturing apparatus 1 can radiate light to the living body 100 from a position closer to the living body 100 than when light is directly radiated from the light emitting unit 52 toward the living body 100. Thereby, the image capturing apparatus 1 can radiate light toward a predetermined position of the living body 100 with a larger incident angle in relation to the living body surface than when light is directly radiated from the light emitting unit 52.

As described above, the image capturing apparatus 1 prevents a reflected light reflected on a living body surface from entering the lens 87, by radiating light to the living body surface with a larger incident angle than when light is radiated on the living body surface from the main body 50. Thereby, the image capturing apparatus 1 can capture a living body image with reduced noise. In this way, the image capturing apparatus 1 can obtain image information from which biometric information of a living body can be acquired preferably.

Also, the support 10 that supports the living body 100 at a right position radiates a predetermined light from the light radiating section 17 toward the living body. Thereby, the image capturing apparatus 1 can support the living body 100 at a right position and preferably radiate light toward the living body 100, when the support 10 attached to the main body 50 is selected from among supports of different shapes and changed to a support (not depicted) of a shape appropriate for the living body 100, for example.

Note that the light 110 that has passed through the polarization filter 80 is linearly-polarized light (P wave) of a vibration direction in parallel with an entrance surface of the living body 100 (the horizontal direction of FIG. 6, the arrow direction of FIG. 7, and the thickness direction of the rear wall 13). The linearly-polarized light (P wave) of the vibration direction in parallel with the entrance surface has a smaller reflectance as compared with linearly-polarized light (S wave) of a vibration direction in parallel with the entrance surface, so as to reduce the light amount of reflected light. Thereby, the image capturing apparatus 1 captures an image of the living body with reduced noise.

Note that the linearly-polarized light (P wave) of the vibration direction in parallel with the entrance surface is 0 in reflectance, when the incident angle to the entrance surface is a Brewster's angle. Thus, the image capturing apparatus 1 captures an image of the living body with less noise, by radiating light in such a manner that the incident angle to the living body surface is a Brewster's angle.

Note that the image capturing apparatus 1 may coat a part of the outer circumferential surface of the rear wall 13, the front wall 14, and the two side walls 15 with an infrared reflection film or an infrared absorption film. The image capturing apparatus 1 prevents infrared light (noise other than a reflected light) from entering the lens 87 from the outer circumferential surface side of the support 10, by coating with the infrared reflection film or the infrared absorption film. Thereby, the image capturing apparatus 1 captures an image of the living body 100 with reduced noise.

Note that, in the above description, the support guides light by total reflection but is not limited thereto. For example, the support 10 may guide light by specular reflection with a mirror or the like.

Although light is guided in and radiated from the rear wall 13, light is guided in and radiated from the front wall and the two side walls 15 in the same way.

### Second Embodiment

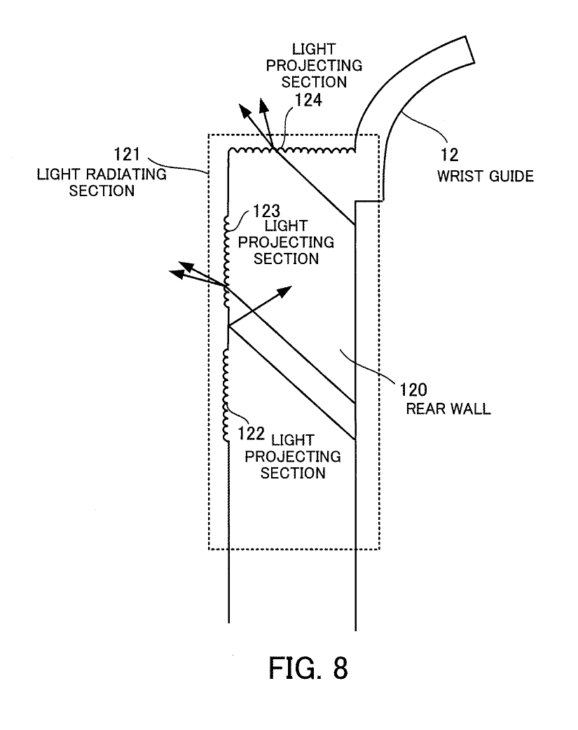

Next, an exemplary variant of the light radiating section will be described with reference to FIG. 8. FIG. 8 is a cross-sectional view of the rear wall in the second embodiment, which is cut in the same cross-section as FIG. 5. Note that the second embodiment can be the same configuration as the first embodiment, except the light radiating section. Note that the same components as the first embodiment will be denoted with the same reference signs.

The light radiating section 121 is provided at a predetermined position on the inner circumferential surface of the top face side of the rear wall 120 and at the upper end of the rear wall 120. That is, the light radiating section 121 is provided at a position away from the bottom face side of the rear wall 120 toward the top face side. The light radiating section 121 includes light projecting sections 122, 123, and 124 that project light guided from the bottom face side. The light projecting sections 122 and 123 are irregularities formed on the inner circumferential surface of the rear wall 120, and scatter and project light guided through the inner portion of the rear wall 120. The light projecting section 124 is an irregularity formed on the top face of the rear wall 120, and scatters and projects light guided through the inner portion of the rear wall 120. The irregularities are formed as grooves and protrusions on the surface of the rear wall 120. Note that the irregularities are examples of the light scattering means, and may be silk printing of reflection dots, for example. Of the light that is incident on the light projecting sections 122, 123, and 124, the light that does not satisfy a total reflection condition exits from the light projecting sections 122, 123, and 124.

Note that the light projecting sections 122, 123, and 124 are examples and are not limited thereto. For example, the light radiating section 121 can adjust how light is radiated to a living body, by changing the number of light projecting sections, the shapes of the light projecting sections, and the formation interval of the light projecting sections.

Also, the light radiating section 121 can adjust how light is radiated to a living body, by changing the shapes of the light projecting sections and the formation interval of the light projecting sections with respect to each light projecting section. Also, the light radiating section 121 may be formed on the entire area of the rear wall 120.

Note that the light radiating section 121 is not limited to the one including all of the light projecting sections 122, 123, and 124, but may include either the light projecting sections 122 and 123 or the light projecting section 124. Although the light radiating section 121 in the rear wall 120 has been described, the light radiating section 121 is configured in the same way in the front wall and the two side walls (not depicted). In this case, the image capturing apparatus may differentiate how light is radiated at the rear wall 120, the front wall, and the two side walls from each other. Note that the shapes of the light projecting sections of the first and second embodiments can be combined as appropriate.

Although light is guided in and radiated from the rear wall 120, light may be guided in and radiated from the front wall and the two side walls in the same way.

### Third Embodiment

In the first embodiment, the image capturing apparatus 1 is configured in such a manner that the support 10 can be attached to the main body 50 at a right position by means of the contact guide 16. On the other hand, in the third embodiment, the image capturing apparatus allows the support to be attached to the right position of the main body, by engaging recessed portions formed at the end portions of the bottom face side of the rear wall, the front wall, the two side walls, with convex portions formed at the housing top face in order to restrict movement of the support in the frontward, rearward, leftward, and rightward directions relative to the main body, as well as by the contact guide 16.

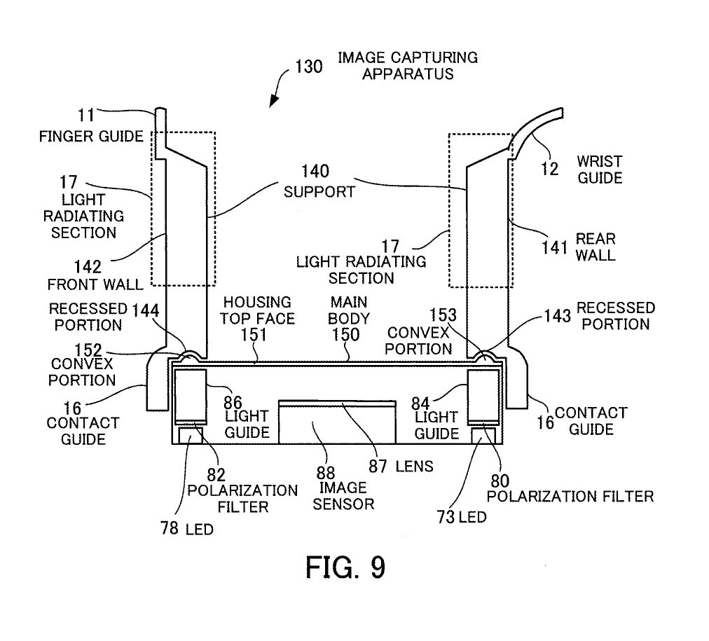

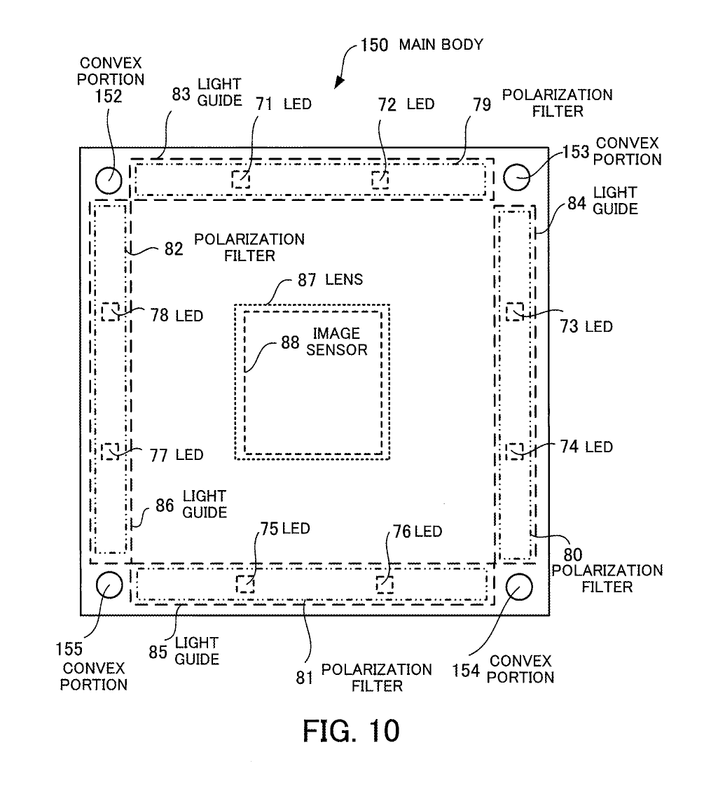

Note that, the third embodiment is the same as the first embodiment, except for engagement between the support and the main body. An exemplary variant of engagement between the support and the main body will be described with reference to FIGS. 9 and 10. FIG. 9 is a cross-sectional view of the image capturing apparatus in the third embodiment, which is cut in the same cross-section as FIG. 6. FIG. 10 is a top perspective view of the main body of the third embodiment. Note that the same components as the first and second embodiments will be denoted with the same reference signs.

An image capturing apparatus 130 includes a support 140 and a main body 150. The main body 150 includes convex portions 152 to 155 at four corners of the housing top face 151. The convex portions 152 to 155 are provided at the four corners so as not to overlap light guides 83 to 86, and thus do not have an influence (such as refraction) on light. Note that the convex portions 152 to 155 are not necessarily provided at all of the four corners. Also, the convex portions 152 to 155 may be provided along the periphery of the housing top face 151.

The support 140 includes a rear wall 141, a front wall 142, and two side walls (not depicted). A recessed portion 143 is provided at an intersection between the rear wall 141 and one of the side walls (not depicted) at the end portion of the bottom face side, in order to engage with the convex portion 153 and attach the support 140 at a right position on the main body 150. Also, a recessed portion (not depicted) is provided at an intersection between the rear wall 141 and the other of the side walls (not depicted) at the end portion of the bottom face side, in order to engage with the convex portion 154 and attach the support 140 at the right position on the main body 150.

The recessed portion 144 is provided at an intersection between the front wall 142 and the one of the side walls (not depicted) at the end portion of the bottom face side, in order to engage with the convex portion 152 and attach the support 140 at the right position on the main body 150. Also, the recessed portion (not depicted) is provided at an intersection between the front wall 142 and the other of the side walls (not depicted) at the end portion of the bottom face side, in order to engage with the convex portion 155 and attach the support 140 at the right position on the main body 150.

Thereby, the recessed portions and the convex portions allow the support 140 to be attached at the right position of the main body 150 and restrict movement of the support 140 in the frontward, rearward, leftward, and rightward directions relative to the main body 150, when the support 140 is attached to the main body 150. Note that the convex portions may be formed in the support 140, and the recessed portions may be formed in the main body 150.

### Fourth Embodiment

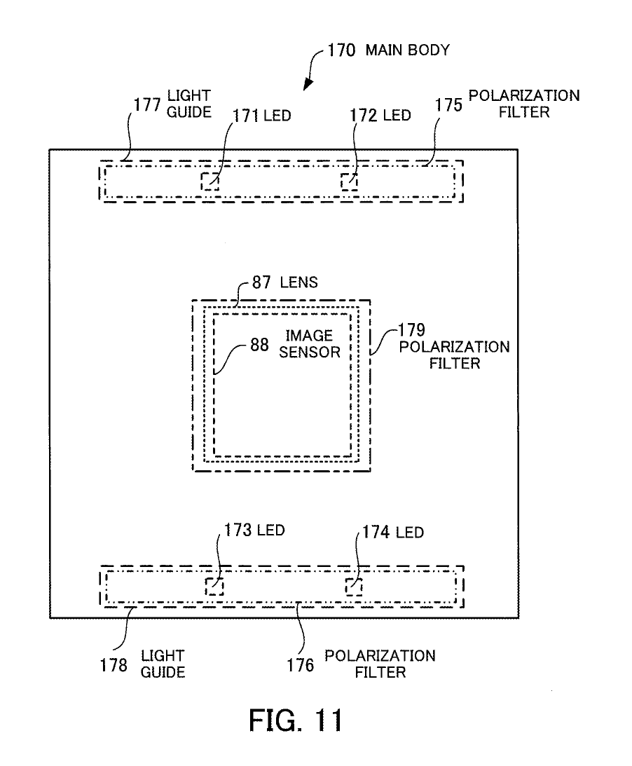

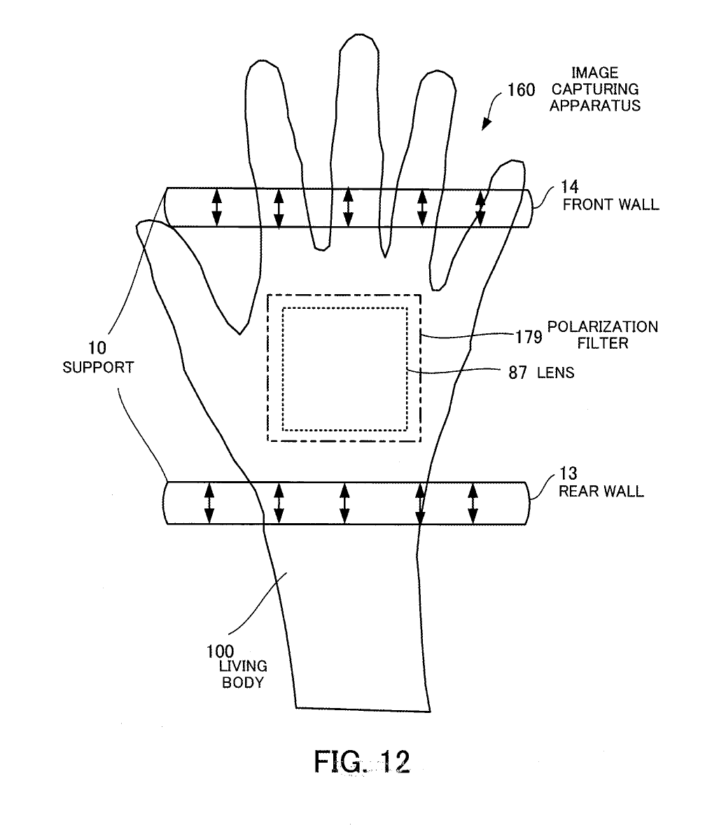

In the first embodiment, the light emitting unit surrounds the circumference of the imaging unit 51 along the four sides of the housing. On the other hand, in the fourth embodiment, the light emitting units are provided on two of the four sides which faces each other. The light emitting units of the fourth embodiment will be described with reference to FIGS. 11 and 12. FIG. 11 is a top perspective view of the main body in the fourth embodiment. FIG. 12 is a top perspective view of the image capturing apparatus in the fourth embodiment. Note that the same components as the first to third embodiments will be denoted with the same reference signs. The image capturing apparatus 160 includes a main body 170 and a support 10.

The main body 170 contains a lens 87, an image sensor 88, a polarization filter 179, LEDs 171 to 174, polarization filters 175 and 176, and light guides 177 and 178.

The lens 87, the image sensor 88, and the polarization filter 179 are components of the imaging unit. The image sensor 88 faces toward an image capturing target via the lens 87 and the polarization filter 179. The LEDs 171 to 174, the polarization filters 175 and 176, and the light guides 177 and 178 are components of the light emitting units. Light emitted from the LEDs 171 to 174 passes through the polarization filters 175 and 176 and the light guides 177 and 178 and exits from the housing top face.

The LEDs 171 to 174 sandwich the image sensor 88 at two sides, each of which is provided with two LEDs. The LEDs 171 to 174 are provided along the two sides of the housing which extend in the horizontal direction. Each of the LEDs 171 to 174 emits light to the housing top face side and ultimately to a living body. Note that the number of LEDs provided in the main body 170 is an example and is not limited thereto.

The polarization filters 175 and 176 are provided at the housing top face side of the LEDs. The polarization filter 175 is provided at the housing top face sides of the LED 171 and the LED 172. The polarization filter 176 is provided at the housing top face sides of the LED 173 and the LED 174. Note that a polarization filter may be provided for each LED.

The polarization filters 175 and 176 receive incoming light and transmit linearly-polarized light having a vibration component in a predetermined direction. The transmission axes of the polarization filters 175 and 176 are the vertical direction of FIG. 11 (the arrow direction of FIG. 12). Thus, the polarization filters 175 and 176 receive incoming light and transmit linearly-polarized light having a vibration component in the vertical direction of FIG. 11 (the arrow direction of FIG. 12).

The light guides 177 and 178 are provided at the housing top face sides of the polarization filters. The light guide 177 is provided at the housing top face side of the polarization filter 175. The light guide 178 is provided at the housing top face side of the polarization filter 176. With these light emitting units, light of a vibration direction in parallel with an entrance surface of the living body 100 (the vertical direction of FIG. 11, the arrow direction of FIG. 12) can be radiated to the living body 100.

The polarization filter 179 is provided at the housing top face side of the lens 87. The transmission axis of the polarization filter 179 is in a direction orthogonal to the transmission axes of the polarization filters 175 and 176 (the horizontal direction of FIG. 12). Thereby, the polarization filter 179 blocks a reflected light having a vibration direction of the arrow direction of FIG. 12. Thus, the polarization filter 179 blocks a reflected light which is reflected on a living body surface and whose vibration direction is unchanged. Thereby, the image capturing apparatus 160 captures an image of the living body 100 with reduced noise. In this way, the image capturing apparatus 160 can obtain image information from which biometric information of the living body 100 can be acquired preferably.

Although in the above description the light emitting units are configured in such a manner that the image sensor 88 is sandwiched by two sides, the light emitting units are not limited thereto but may be provided at only one of the two sides.

### Fifth Embodiment

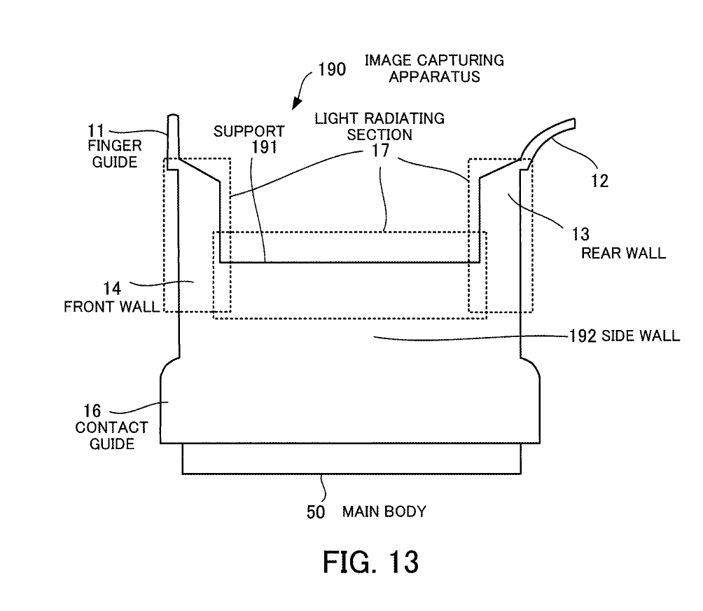

Next, an exemplary variant of the support will be described with reference to FIG. 13. FIG. 13 is an exterior view of the image capturing apparatus in the fifth embodiment. Note that the same components as the first to fourth embodiments will be denoted with the same reference signs.

The image capturing apparatus 190 includes a support 191 and a main body 50. The support 191 includes a rear wall 13, a front wall 14, and two side walls 192, each of which stands from a bottom face. The side walls 192 have heights lower than the rear wall 13 and the front wall 14.

For example, the light radiating sections 17 of the two side walls 192 are provided only at the top faces of the side walls 192. The light radiating section 17 of the rear wall 13 is provided at a predetermined position of the inner circumferential surface of the top face side of the rear wall 13 and at the top face of the rear wall 13. The light radiating section 17 of the front wall 14 is provided at a predetermined position of the inner circumferential surface of the top face side of the front wall 14 and at the top face of the front wall 14.

In the image capturing apparatus 190, the support 191 allows light emitted by the main body 50 to enter the end portions of the bottom face sides of the rear wall 13, the front wall 14, and the two side walls 192, and guides the light from the bottom face sides toward the top face sides, and radiates the light toward a living body 100 from the light radiating sections 17. Thus, the image capturing apparatus 190 can radiate light toward the living body 100 from a position closer to the living body 100 than when light is directly radiated to the living body 100 from the light emitting unit 52. Thereby, the image capturing apparatus 190 can radiate light to a predetermined position of the living body 100 with a larger incident angle in relation to the living body surface than when light is directly radiated from the light emitting unit 52.

As described above, the image capturing apparatus 190 prevents a reflected light reflected on a living body surface from entering the lens 87, by radiating light to a living body surface with a larger incident angle than when light is radiated on the living body surface from the main body 50. Thereby, the image capturing apparatus 190 captures an image of the living body 100 with reduced noise. In this way, the image capturing apparatus 190 can obtain image information from which biometric information of the living body 100 can be acquired preferably.

Note that the two side walls 192 may be removed so that the support 191 is formed with the rear wall 13 and the front wall 14 that stand from the bottom face. In this case, light may be radiated to the living body 100 as described in the fourth embodiment.

### Sixth Embodiment

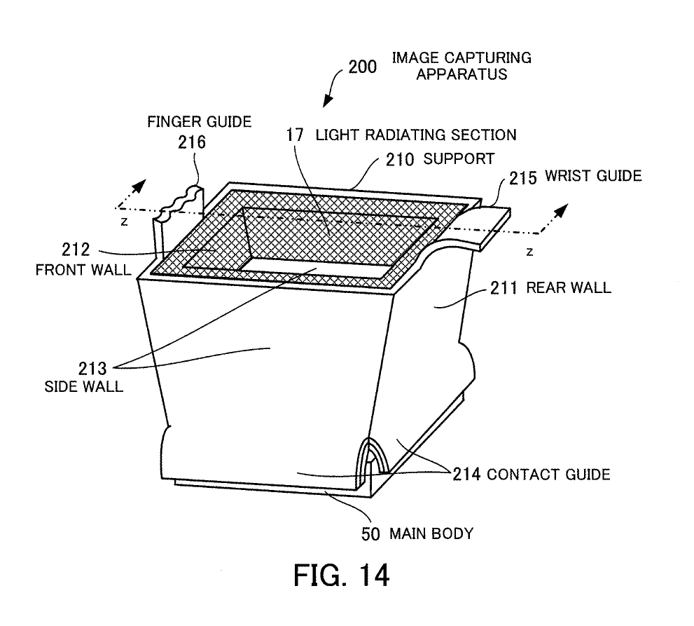

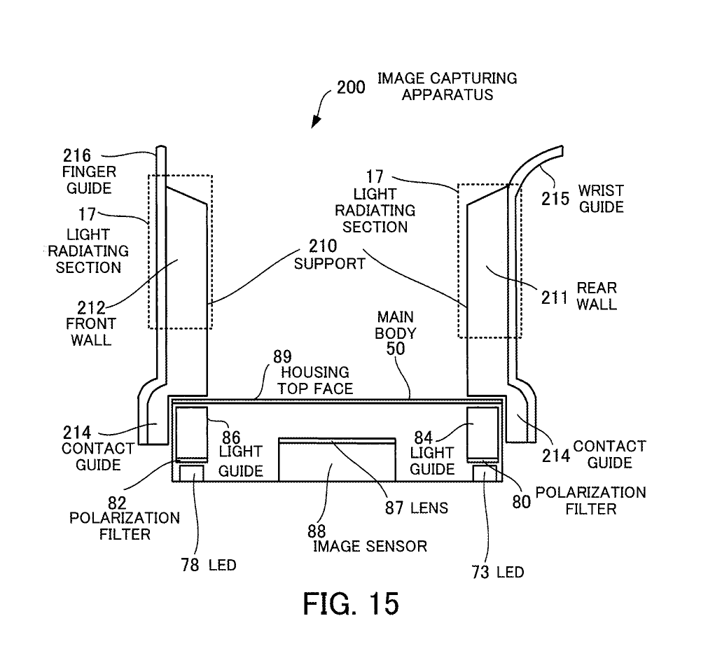

Next, an exemplary variant of the support will be described with reference to FIGS. 14 and 15. FIG. 14 is a perspective view of the image capturing apparatus in the sixth embodiment. FIG. 15 is a cross-sectional view of the image capturing apparatus in the sixth embodiment, which is cut along the z-z line of FIG. 14.

The image capturing apparatus 200 includes a support 210 and a main body 50. The support 210 includes a rear wall 211, a front wall 212, and two side walls 213, each of which stands from the bottom face. Also, the support 210 includes contact guides 214 on the outer circumferential surfaces of the bottom face sides of the rear wall 211, the front wall 212, and the two side walls 213. Each of the rear wall 211, the front wall 212, the two side walls 213, and the contact guides 214 has a two-layer structure consisting of an inner circumferential surface side (a light guide member) made of a transparent plastic, such as acrylic, and an outer circumferential surface side made of an acrylonitrile butadiene styrene (ABS) plastic or the like.

The rear wall 211 includes a wrist guide 215 that guides a wrist to an appropriate mounting position at the top face side. The wrist guide 215 is formed of an ABS plastic. The front wall 212 includes a finger guide 216 that guides fingers to an appropriate mounting position at the top face side. The finger guide 216 is formed of an ABS plastic. Note that each of the wrist guide 215 and the finger guide 216 may have a two-layer structure consisting of a transparent plastic and an ABS plastic.

As described above, the rear wall 211, the front wall 212, the two side walls 213, and the contact guides 214 are formed in two-layer structures, and therefore the support 210 can increase its strength (heat resistance, impact resistance, etc.) as well as prevent an external light from entering the lens 87 from the outer circumference. Thereby, the image capturing apparatus 200 captures an image of the living body 100 with reduced noise. In this way, the image capturing apparatus 200 can obtain image information from which biometric information of the living body 100 can be acquired preferably.

### Seventh Embodiment

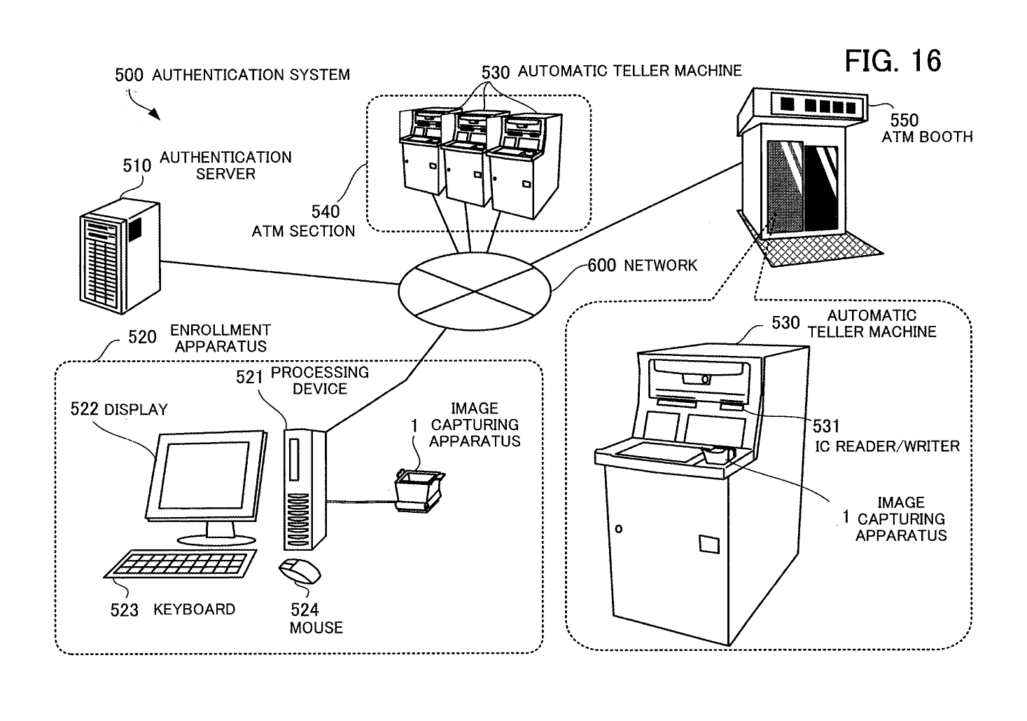

Next, an authentication system utilizing the image capturing apparatuses described in the first to sixth embodiments will be described with reference to FIG. 16. FIG. 16 illustrates an exemplary application of the image capturing apparatus in the seventh embodiment.

The authentication system 500 is one of information processing systems that identify and authenticate individuals by recognizing features of living bodies, in order to authenticate customers in a bank system or the like, for example. The authentication system 500 includes an enrollment apparatus 520, a plurality of automatic teller machines 530, an information processing apparatus such as an authentication server 510, and a network 600.

The authentication server 510 stores identification information for identifying an individual and verification information (template) enrolled in advance before biometric authentication, in association with each other. The identification information for identifying an individual is a unique ID (IDentification) assigned to a user directly (for example, user number) or indirectly (for example, account number).

One or a plurality of automatic teller machines 530 are installed at an automated teller machine (ATM) section 540 or an ATM booth 550 inside a financial institution. An automatic teller machine 530 is one of authentication apparatuses that execute biometric authentication when authenticating a user before financial transaction. The automatic teller machine 530 includes an integrated circuit (IC) reader/writer 531 and an image capturing apparatus 1 (130, 160, 190, and 200). The automatic teller machine 530 executes user authentication on the basis of verification information that is associated with the identification information that the IC reader/writer 531 reads from an IC card (for example, a cash card with an IC chip) of a user and user's biometric information that is extracted from a living body image captured by the image capturing apparatus 1 (130, 160, 190, and 200).

The enrollment apparatus 520 is provided at a teller window of a bank for example, for the purpose of enrolling templates of users in accordance with an instruction or operation of a teller. The enrollment apparatus 520 includes a processing device 521, a display 522, and an image capturing apparatus 1 (130, 160, 190, and 200), as well as a keyboard 523 and a mouse 524 if needed. The image capturing apparatus 1 (130, 160, 190, and 200) includes a communication interface to communicate with the enrollment apparatus 520. The image capturing apparatus 1 (130, 160, 190, and 200) captures an image of a living body of a user. The image capturing apparatus 1 (130, 160, 190, and 200) outputs biometric information acquired from a captured living body image or verification information generated on the basis of the biometric information. The image capturing apparatus 1 (130, 160, 190, and 200) records the biometric information or the verification information in at least one of a memory unit of the processing device 521, a memory unit of the authentication server 510, and a memory unit of the IC card of a user.

Although the authentication system 500 illustrated in FIG. 16 includes the enrollment apparatus 520 having the processing device 521 connected to the image capturing apparatus 1 (130, 160, 190, and 200), the authentication system 500 may be replaced by an authentication apparatus that executes biometric authentication on the basis of biometric information or verification information, when templates of the verification information are enrolled in advance. For example, in the automatic teller machine 530, the image capturing apparatus 1 (130, 160, 190, and 200) may be replaced by a sensor unit 532.

Although in the above embodiments a palm vein image is captured as biometric information for use in authentication, the image capturing apparatus 1 (130, 160, 190, and 200) may capture a palm print image.

The image capturing apparatus and the authentication apparatus are capable of capturing a living body image with reduced noise.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. An image capturing apparatus comprising:
a main body including an imaging unit that captures an image of a living body; and
a support that contacts the main body at one side and supports the living body at a predetermined position relative to the imaging unit at another side,
wherein the main body includes a light emitting unit that emits light for the living body, and
the support includes:
an entrance section where the light enters the one side,
a light radiating section that radiates the light toward the living body at a position away from the entrance section toward the other side, and
a light guiding section that guides the light from the entrance section to the light radiating section.

2. The image capturing apparatus according to claim 1, wherein
the support includes a standing wall that stands from the one side toward the other side, and
the standing wall includes the entrance section, the light guiding section, and the light radiating section.

3. The image capturing apparatus according to claim 2, wherein
the standing wall includes a supporting section that supports the living body.

4. The image capturing apparatus according to claim 2, wherein
the standing wall includes the light radiating section on an end surface of the other side.

5. The image capturing apparatus according to claim 2, wherein
the standing wall includes a supporting section that supports the living body, and the light radiating section on an end surface of the other side.

6. The image capturing apparatus according to claim 1, wherein
the light emitting unit includes a polarization filter that transmits linearly-polarized light included in the light.

7. The image capturing apparatus according to claim 1, wherein
the support is attachable to and detachable from the main body.

8. The image capturing apparatus according to claim 2, wherein
the support includes the standing wall surrounding the imaging unit, and
the standing wall includes a blocking section that blocks light from outside, on an outer circumferential surface of the standing wall.

9. An authentication apparatus comprising:
a main body including an imaging unit that captures an image of a living body;
a support that contacts the main body at one side and supports the living body at a predetermined position relative to the imaging unit at another side; and
an authentication unit that executes authentication by using biometric information included in information output from the imaging unit,
wherein the main body includes a light emitting unit that emits light for the living body, and
the support includes:
an entrance section where the light enters the one side,
a light radiating section that radiates the light toward the living body at a position away from the entrance section toward the other side, and
a light guiding section that guides the light from the entrance section to the light radiating section.
